(19) United States
(12) Patent Application Publication
(10) Pub. No.: US 2024/0008453 A1
(43) Pub. Date: Jan. 11, 2024

(54) SYSTEM FOR INDUCING PET MOVEMENT IN METAVERSE ENVIRONMENT

(71) Applicants: MAX.K KOREA Inc., Seoul (KR); Max K LLC, Houston, TX (US)

(72) Inventors: Kibun Max KOO, Houston, TX (US); Sang Min AN, Seoul (KR)

(73) Assignees: MAX.K KOREA Inc., Seoul (KR); Max K LLC, Houston, TX (US)

(21) Appl. No.: 18/181,980

(22) Filed: Mar. 10, 2023

(30) Foreign Application Priority Data
Jul. 5, 2022 (KR) 10-2022-0082306

(51) Int. Cl. A01K 15/02; G06T 11/00; G06T 7/70; A01K 29/00

(52) U.S. Cl. CPC A01K 15/027; G06T 11/00; G06T 7/70; A01K 29/005; G06T 2200/24

(57) ABSTRACT

The present disclosure relates to a system and methods for inducing pet movement in a metaverse environment. More particularly, the system is configured to enable a user to monitor an action of a pet and perform an interaction with the corresponding pet by using tools in various shapes and types including a toy capable of inducing an action of the pet, thereby being capable of satisfying an exercise amount required for the pet.

### CROSS REFERENCE TO RELATED APPLICATION

This application claims priority to Korea Patent Application No. 10-2022-0082306 filed on Jul. 5, 2022, the content of which is expressly incorporated by reference in its entirety.

### TECHNICAL FIELD

The present disclosure relates to a system and methods for inducing pet movement in a metaverse environment. More particularly, the system is configured to enable a user to monitor an action of a pet and perform an interaction with the corresponding pet by using tools in various shapes and types including a toy capable of inducing an action of the pet, thereby being capable of satisfying an exercise amount required for the pet.

### BACKGROUND

Many problems can be solved if a life or an activity in a real world is capable of being connected to a virtual world. Currently, a metaverse system in which a life or an activity in a real world is capable of being substituted by utilizing an avatar of a user in a virtual world is being actively introduced in all fields. As an example, a metaverse environment is capable of being constructed by receiving sensing signal from various sensors, and such a concept and a method are known in a conventional art. However, various methods are capable of existing in a structure connecting a real environment to a virtual environment in the metaverse environment.

However, since a method for maintaining a health of a pet by inducing the movement of the pet through a game or play in the metaverse environment has not yet been developed, the user is required to go out for a walk or movement with the pet every time in real life. However, it is very uncomfortable and difficult for the user who needs to take a rest usually after holidays or after work to take a pet outdoors and walk or movement.

Therefore, in a situation in which the user goes to work or the user is located outside, a technology of inducing the movement of the pet through an automatically set scenario such as a game by accessing the metaverse environment is required.

Korean Patent No. 10-2402168 (2022.05.25.) discloses a method of exercising and training a companion animal by using an autonomous driving robot. In the Korean Patent No. 10-2402168, an opening unit that is provided in an autonomous driving robot and is accommodating a snack for a companion animal is partially opened by a control signal of a user terminal or when a time preset by the user terminal is reached; a moving process in which the autonomous driving robot moves according to a driving path set by using biometric information of the companion animal; a detecting process in which location information of the companion animal and distance information of the companion animal from the autonomous driving robot are detected on the basis of a signal received from a sensor module attached to a part of a body of the companion animal; a discharging process in which a smell of the snack for the companion animal is discharged outside the opening unit by operating an air circulation unit provided at the opening unit, the smell being discharged on the basis of the distance information; and a deriving and determining process in which an activity score for assessing an exercise performance ability of the companion animal is derived on the basis of the location information and the amount of feed discharged through the opening unit is determined on the basis of the derived activity score.

Korean Patent No. 10-2402168 provides a method of complementing an insufficient exercise amount by inducing interest in the companion animal in a single-person household even if a guardian is away from home is proposed.

Korean Patent Application No. 10-2018-0110763 (2018.10.11.) discloses a companion animal management service apparatus for an exercise management of the companion animal and a user terminal. In the companion animal management service apparatus: a synchronization unit obtaining and synchronizing medical data of an animal hospital apparatus; an exercise management service unit obtaining user identification information of the user terminal that a guardian of the companion animal uses, the exercise management service unit extracting companion animal information matched with the user identification information from the synchronized medical data, and the exercise management service unit providing the exercise management service suitable for the state of the companion animal to the user terminal on the basis of the extracted companion animal information; and a database in which medical data obtained by the plurality of animal hospital apparatuses is stored.

Korean Patent Application No. 10-2018-0110763 describes synchronizing the animal hospital apparatus with the user terminal that the guardian owns and providing the exercise management service suitable for the state of the companion animal designated as a target of the exercise management on the basis of the synchronized companion animal information is proposed.

There is a need and a desire for improved systems and methods for inducing pet movement.

### BRIEF SUMMARY OF THE EMBODIMENTS

According to an embodiment of the present disclosure, a system is provided for inducing pet movement in metaverse environment, the system including: a metaverse environment generation part configured to generate a metaverse environment by mapping temporal and spatial information for a wearable device worn on a pet and for a movement inducing device inducing an action of the pet existing in a virtual space for a living space of the pet; and a movement inducing part configured to operate the movement inducing device on the basis of a control of a user accessing the generated metaverse environment, wherein the exercise amount required for the pet is satisfied by the user who induces the pet movement in the metaverse environment.

In addition, the metaverse environment generation part is further configured to receive location information for the wearable device of the pet and the movement inducing device, map the received location information to avatars of the pet and the movement inducing device at corresponding locations in the virtual space, and update a temporal change of the location information in real time, thereby synchronizing and connecting the avatars in the virtual space to a living environment where the pet actually lives.

At this time, the location information of the pet and the movement inducing device is coordinate information that checks in which space the pet and the movement inducing device are located among spaces partitioned at predetermined intervals, the coordinate information being checked on the basis of image information captured by a camera in an agent device provided in the living environment where the pet lives. Furthermore, the metaverse environment generation part is configured to update the avatars for the pet and the movement inducing device in real time and to display the avatars at the corresponding location in the virtual space on the basis of the coordinate information of the pet and the movement inducing device received from the agent device.

In addition, the system further includes a pet monitoring part configured to monitor the action of the pet in the metaverse environment, and the action of the pet is synchronized by updating location information of the pet in the metaverse environment through the avatars rather than the image of the pet.

In addition, the system further includes a pet exercise amount measuring part configured to measure the exercise amount of the pet. Furthermore, the movement of the pet is induced by the user in the metaverse environment so that a target exercise amount of the pet is reached on the basis of the measured exercise amount of the pet, thereby satisfying an exercise amount required for the pet. Furthermore, the exercise amount of the pet is calculated by applying changes in coordinates and speeds for the movement of the pet in the metaverse environment, values detected from a gyro sensor and an acceleration sensor, or a combination thereof.

In addition, the movement inducing device includes a house, a toy, play tool, a feed bucket, a microphone, an image display device, a speaker, or a smell generator, and an operation of the movement inducing device is performed by vibration, sound, an image, smell, or a combination thereof.

In addition, the system further includes an exercise scenario setting part configured to set and store at least more than one scenario for the pet movement induction, comprising an operation sequence, an operation method, or a reward offering for each operation of at least more than one movement inducing device located in the virtual space provided for each user; and an exercise scenario providing part configured to provide information for a specific scenario that the user selects among the set scenarios to the movement inducing part, wherein the movement inducing part is further configured to control operating at least one of the movement inducing device according to the specific scenario that the user selects.

In addition, the system further includes an exercise scenario assessing part configured to assess which body part is induced to movement by continuously tracking the movement of the pet in which the movement is induced by the specific scenario according to the location information received from the wearable device; and an exercise scenario modifying part configured to modify a scenario so as to complement an insufficient movement and an insufficient action of the pet on the basis of the assessed results.

In addition, the system further includes a customized exercise scenario setting part configured to set and store a customized scenario comprising the operation sequence, the operation method, or the reward offering of at least one movement inducing device located in the virtual space, according to a direct selection of the user accessing the metaverse environment, wherein the exercise scenario modifying part is configured to modify the customized scenario on the basis of the assessed results of the exercise scenario assessing part for the movement of the pet according to the customized scenario.

In addition, the system is further configured to: control an operation such that the movement inducing device is operated through the movement inducing part according to different scenarios for a plurality of pets on the basis of an operation of the user accessing the metaverse environment; and priorly control the operation of the movement inducing device commonly applied among scenarios performed for each of the plurality of pets, and then control a remaining operation of the movement inducing device separately performed.

In addition, according to an embodiment of the present disclosure, there is provided a method for inducing pet movement in metaverse environment, the method including: generating a metaverse environment by mapping temporal and spatial information for a wearable device worn on a pet and for a movement inducing device inducing an action of the pet existing in a virtual space for a living space of the pet; and inducing a movement by operating the movement inducing device on the basis of a control of a user accessing the generated metaverse environment, wherein the exercise amount required for the pet is satisfied by inducing the pet movement in the metaverse environment.

In addition, the generating of the metaverse environment further comprises: receiving location information for the wearable device of the pet and the movement inducing device, mapping the received location information to avatars of the pet and the movement inducing device at corresponding locations in the virtual space, and updating a temporal change of the location information in real time, thereby synchronizing and connecting the avatars in the virtual space to a living environment where the pet actually lives.

In addition, the method further includes monitoring the action of the pet in the metaverse environment, wherein the action of the pet is synchronized by updating location information of the pet in the metaverse environment through the avatars rather than the image of the pet.

In addition, the method further comprises: measuring the exercise amount of the pet. Furthermore, the movement of the pet is induced by the user in the metaverse environment so that a target exercise amount of the pet is reached on the basis of the measured exercise amount of the pet, thereby satisfying an exercise amount required for the pet. Furthermore, the exercise amount of the pet is calculated by applying changes in coordinates and speeds for the movement of the pet in the metaverse environment, values detected from a gyro sensor and an acceleration sensor, or a combination thereof.

In addition, the method further includes setting an exercise scenario by setting and storing at least more than one scenario for the pet movement induction, comprising an operation sequence, an operation method, or a reward offering for each operation of at least more than one movement inducing device located in the virtual space provided for each user; and providing the exercise scenario by providing information for a specific scenario that the user selects among set scenarios to the inducing of the movement, wherein the inducing of the movement comprises controlling to operate at least one of the movement inducing device according to the specific scenario that the user selects.

In addition, the method further includes in the system inducing the movement of the pet, assessing the exercise scenario by assessing which body part is induced to movement by continuously tracking the movement of the pet in which the movement is induced by the specific scenario according to the location information received from the wearable device; and modifying the exercise scenario by modifying the scenario so as to complement an insufficient movement and an insufficient action of the pet on the basis of the assessed results.

In addition, the method further includes setting and storing a customized exercise scenario comprising the operation sequence, the operation method, or the reward offering of at least one movement inducing device located in the virtual space according to a direct selection of the user accessing the metaverse environment. Furthermore, the modifying of the exercise scenario is configured to modify the customized scenario on the basis of the assessed results of the assessing of the exercise scenario for the movement of the pet according to the customized scenario.

In addition, the method is further configured to: control an operation such that the movement inducing device is operated through the inducing of the movement according to different scenarios for a plurality of pets on the basis of an operation of the user accessing the metaverse environment; and initially control the operation of the movement inducing device commonly applied among scenarios performed for each of the plurality of pets, and then control a remaining operation of the movement inducing device separately performed.

As described above, according to the system of the present disclosure, since the user located in a remote location accesses the metaverse environment and utilizes the tools in various shapes and types comprising the toy and performs an interaction with the corresponding pet according to a game, play, or an automatically set scenario, there is an effect that the body of the pet is kept healthy by satisfying the exercise amount required for the pet.

In addition, in the present disclosure, since the user requests a specific action to the pet by utilizing voice, image, smell, and so on of the user and the user provides a reward such as a feed to the pet when the corresponding pet correctly performs the requested specific action, there is an effect that exercise efficiency of the pet is increased.

In addition, in the present disclosure, since the movement of the pet according to the interaction with the user is continuously tracked and a program that can complement an insufficient movement or an insufficient action of the pet is capable of being proposed through the tracked results, so that there is an effect that an advanced exercise is induced for each pet.

Other aspects, features, and techniques will be apparent to one skilled in the relevant art in view of the following detailed description of the embodiments.

### BRIEF DESCRIPTION OF THE DRAWINGS

The above and other objectives, features, and other advantages of the present disclosure will be more clearly understood from the following detailed description when taken in conjunction with the accompanying drawings.

FIG. 1 is a view illustrating an overall structure of a system for inducing pet movement in a metaverse environment according to an embodiment of the present disclosure.

FIG. 2 is a view specifically illustrating a configuration of a metaverse platform for inducing a pet movement, according to an embodiment of the present disclosure.

FIG. 3 is a view specifically illustrating a wearable device according to an embodiment of the present disclosure.

FIG. 4 is a view specifically illustrating a movement inducing device according to an embodiment of the present disclosure.

FIG. 5 is a view specifically illustrating a pet movement inducing process in the metaverse environment according to an embodiment of the present disclosure.

FIG. 6 is a view illustrating checking of location information (coordinate) of a pet and the movement inducing device in the metaverse environment according to an embodiment of the present disclosure.

FIG. 7 is a flowchart specifically illustrating an operation process of a pet movement inducing method in the metaverse environment according to an embodiment of the present disclosure.

FIG. 8 is a view illustrating an example of a hardware structure of a computer (server) realizing the metaverse platform for inducing the pet movement, according to an embodiment of the present disclosure.

### DETAILED DESCRIPTION OF THE EXEMPLARY EMBODIMENTS

### Overview and Terminology

Hereinafter, a preferred embodiment of a system for inducing pet movement in a metaverse environment of the present disclosure will be described in detail with reference to the accompanying drawings. The same reference numerals shown in each drawing indicate the same members. In addition, specific structural or functional descriptions of the embodiments of the present disclosure are exemplified only for the purpose of describing the embodiments according to the present disclosure, and unless defined otherwise, all terms used herein, comprising technical or scientific terms, have the same meaning as commonly understood by those skilled in the art to which the present disclosure belongs. Terms such as those defined in a commonly used dictionary should be interpreted as having a meaning consistent with the meaning in the context of the related art, and should not be interpreted as an ideal or excessively formal meaning unless explicitly defined in the present specification.

The present disclosure has been made keeping in mind the above problems occurring in the related art, and an objective of the present disclosure is to provide a system capable of satisfying an insufficient exercise amount of a pet by allowing a user to interact with the pet in a metaverse environment. In addition, another objective of the present disclosure is to provide a system capable of satisfying a required exercise amount by allowing a user to access a metaverse environment, to monitor an action of a pet, and to move the pet by using tools in various shapes and types comprising a toy to play a game or play or an automatically set scenario on the basis of the monitoring results. In addition, still another objective of the present disclosure is to provide a system configured such that a user requests a specific action to a pet by utilizing voice, image, smell, and so on of the user in a metaverse environment and the user provides a reward such as a food or compliment (the favorite music, image, sound, and so on of the pet) when the corresponding pet correctly performs the specific action, thereby being capable of increasing exercise effect of the pet.

In addition, yet another objective of the present disclosure is to provide a system configured to check which part of the body was mainly induced to movement by continuously tracking a movement of the pet caused by the interaction with a user, thereby being capable of proposing an exercise program that can complements an insufficient movement or an insufficient action. In addition, yet another objective of the present disclosure is to provide a system capable of assessing exercise results of a pet according to a preset scenario, the system being capable of realizing an advancement of the exercise of each pet by modifying the scenario according to the assessed results.

The present disclosure is intended to provide a method of maintaining a body of a pet healthy in which the user is capable of performing an interaction with a pet by utilizing tools in various shapes and types comprising a toy or by an automatically set scenario, thereby satisfying the exercise amount required for the pet.

In addition, the present disclosure is intended to provide a method capable of increasing exercise efficiency of the pet by utilizing voice, image, smell, and so on of the user, the method being capable of inducing an advanced exercise for each pet by proposing a program that can complements an insufficient movement and an insufficient action of the pet by tracking the movement of the pet according to the interaction with the user.

Actually, by using the present disclosure, an objective of enjoying leisure while the user interacts with the pet is achieved, and the movement of the pet is induced during performing the objective, so that an additional effect of keeping the body of the pet healthy is capable of being realized.

According to the present disclosure, the user accessing the metaverse environment utilizes tools in various shapes and types comprising the toy and also utilizes voice, image, smell, and so on of the user so as to interact with the corresponding pet, the exercise amount required for the pet is capable of being satisfied. Furthermore, in the present disclosure, an advanced exercise is capable of being induced for each pet by complementing an insufficient movement and an insufficient action of the pet through the tracking of the movement of the pet according to the interaction with the user.

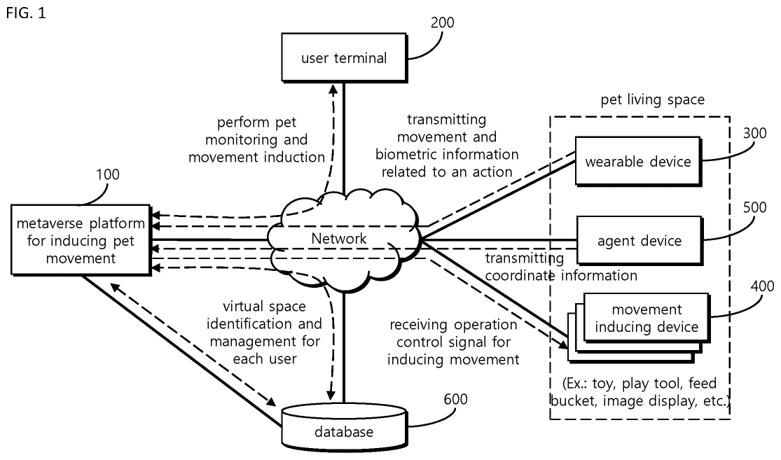

FIG. 1 is a view illustrating an overall structure of a system for inducing pet movement in a metaverse environment according to an embodiment of the present disclosure.

As illustrated in FIG. 1, a system for inducing pet movement according to an embodiment of the present disclosure includes a metaverse platform 100 for inducing a pet movement (hereinafter, referred to as a metaverse platform), a user terminal 200, a wearable device 300, a movement inducing device 400, an agent device 500, and a database 600.

The metaverse platform 100 generates a virtual space for each user, the virtual space reflecting an environment which includes a pet and at least one movement inducing device and in which the pet lives, and the metaverse platform 100 stores the virtual space in a database and manages the virtual space. Furthermore, the metaverse platform 100 allows each user to interact with each pet in a metaverse environment, thereby satisfying the insufficient amount of exercise of the pet positioned in an actual living space.

That is, the user accesses the metaverse environment of the user provided by the metaverse platform 100, and monitors the current state of the pet. Through monitoring results, the user attracts the attention of the pet and makes the pet move by using the movement inducing device 400 constituted of various shapes and types of tools including toys, thereby satisfying the insufficient amount of exercise of the pet. At this time, a metaverse environment built on the basis of a family house (that is, a home) is described as an example of the metaverse environment of the present disclosure, but is not limited thereto, and is capable of including an outdoor space.

In addition, the metaverse platform 100 is capable of utilizing voice, image, smell, and so on of the user to smoothly induce the movement of the pet. Furthermore, the metaverse platform 100 is capable of providing a reward such as food when the pet accurately performs a requested specific action (that is, moving to the movement inducing device).

In addition, in order to induce the movement of the pet, the metaverse platform 100 allows the user to directly select the movement inducing device 400 or to select any one exercise scenario among preset exercise scenarios according to a specific purpose (for example, weight loss, specific part training, simple exercise, and so on).

In addition, the metaverse platform 100 receives information of the pet moving along the movement inducing through the movement inducing device 400 and receives information for a location of the movement inducing device 400 from the agent device 500. Furthermore, a temporal change of avatars for the pet and the movement inducing device 400 that are mapped in the virtual space are capable of being updated in real time with reference to the information for the location. Thus, the user is capable of inducing the movement of the pet while continuously interacting with the pet.

In addition, the metaverse platform 100 is capable of continuously tracking the movement of the pet by the user interacting with the pet in the metaverse environment, is capable of assessing which part of the movement of the pet is mainly induced, and is capable of modifying an exercise scenario on the basis of the assessed result so that an insufficient movement or an insufficient action is complemented.

In addition, the metaverse platform 100 is capable of supporting the user such that an operation order, an operating method, a reward, and so on of each movement inducing device 400 that the user directly selects is set as a customized exercise scenario. Furthermore, by an assessed result of the customized exercise scenario, a program complementing an insufficient movement or an insufficient action is capable of being provided to a corresponding user.

The user terminal 200 is a communication terminal such as a smartphone, a tablet, or the like owned by the user (that is, an owner or a family member) who manages the pet. Furthermore, the user terminal 200 performs a communication connection to the metaverse platform 100, and monitors an action or a state of the pet positioned in the family house in the metaverse environment. That is, by an application executed on the user terminal 200, a current activity and a health status of the pet displayed as the avatar in the virtual space is checked.

In addition, on the basis of a selection of the user who monitored the state of the pet, the user terminal 200 transmits operation information of an operation (for example, generation of sound or vibration, display of image, and so on) of various movement inducing devices 400 for inducing the movement of the pet or selection information of a specific exercise scenario to the metaverse platform 100.

In addition, the user terminal 200 receives the assessed result according to the movement of the pet induced in the metaverse platform from the metaverse platform 100, and the user terminal 200 is capable of checking how much the pet has exercised, which body part of the pet has been trained, and the like.

The wearable device 300 is configured to be used by fastening the wearable device 300 to body parts such as neck, legs, torso, and so on of the pet, and includes various sensors such as an acceleration sensor, a gyro sensor, a biometric sensor, and so on.

In addition, the wearable device 300 measures movement information and biometric information that are caused by the movement of the pet according to an attraction induction of the various movement inducing devices 400 disposed in the family house, and transmits the movement information and the biometric information that are measured to the metaverse platform 100.

The movement inducing device 400 is capable of communicating with the metaverse platform 100, and is constituted of various shapes and types of tools for inducing the movement of the pet. Furthermore, the movement inducing device 400 includes a pet house fixed to a predetermined position, an image display device and a speaker for outputting an image, a smell generator, a feed bucket capable that is capable of being moved, a dinnerware, a toy, play tool, and so on.

In addition, the movement inducing device 400 is provided with an operation unit capable of generating vibration, sound, an image, smell, and so on by an operation control signal that is provided from the metaverse platform 100. That is, by controlling the metaverse platform 100 according to the control of the user accessing the metaverse environment, vibration, sound, an image, smell, and so on are generated so that the attraction of the pet is induced, thereby moving the pet.

The agent device 500 includes at least one camera and is mounted in a living space of the pet. Furthermore, the agent device 500 transmits location information of the pet moved by the induction of the movement inducing device 400 and also transmits location information of the movement inducing device 400 moved by the pet to the metaverse platform 100.

That is, in a state in which the living space of the pet is partitioned at predetermined intervals up, down, left, and right, image information captured by the camera is analyzed and which space the pet and the movement inducing device 400 are located among the spaces partitioned at the predetermined intervals is checked, and coordinate information for the checked space is provided to the metaverse platform 100. Therefore, in the metaverse platform 100, the avatars for the pet and the movement inducing device 400 positioned at the corresponding location in the virtual space are capable of being updated and displayed in real time.

At this time, an agent device disposed in the living space of the pet is described as an example of the agent device 500, but is not limited thereto, and the agent device 500 is capable of being provided in the metaverse platform 100. However, since image information captured by the camera is required to be received and analyzed in the metaverse platform 100, the agent device 500 is not recommended to be provided in the metaverse platform 100 due to problems such as data transmission traffic, a processing speed, and so on.

In addition, the agent device 500 is capable of including a plurality of cameras so that an actual living space of the pet is covered without blind spots. At this time, the agent device 500 is capable of communicating with each of the cameras in a wired manner and a wireless manner.

The database 600 manages and stores the virtual space (that is, a space that reproduces a real environment where the pet lives) generated for each user in the metaverse platform 100, and stores and manages various exercise scenarios for inducing the movement of the pet.

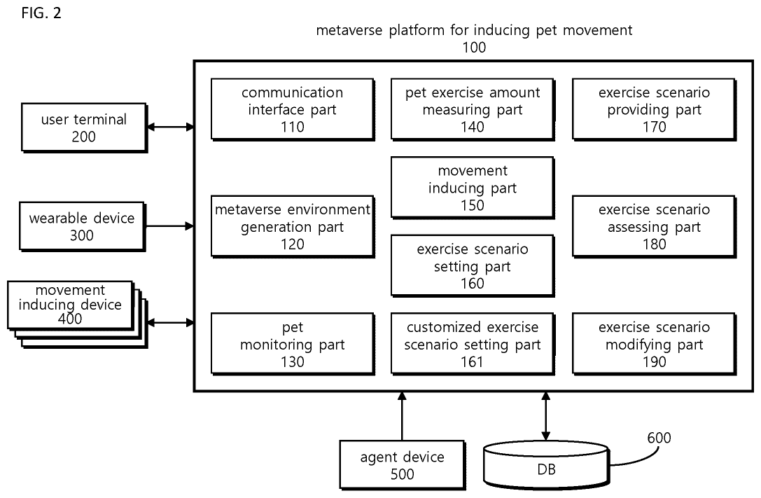

FIG. 2 is a view specifically illustrating a configuration of a metaverse platform for inducing a pet movement, according to an embodiment of the present disclosure.

As illustrated in FIG. 2, the metaverse platform 100 includes a communication interface part 110, a metaverse environment generation part 120, a pet monitoring part 130, a pet exercise amount measuring part 140, a movement inducing part 150, an exercise scenario setting part 160, a customized exercise scenario setting part 161, an exercise scenario providing part 170, an exercise scenario assessing part 180, and an exercise scenario modifying part 190, and so on.

The communication interface part 110 performs communication connections with the user terminal 200, the wearable device 300, the movement inducing device 400, and the agent device 500, thereby performing a data communication related to the pet movement inducing in the metaverse environment.

For example, the communication interface part 110 continuously provides information for a current exercise amount, biometric information, an action, and so on of the pet to the user terminal 200 accessing the metaverse environment, and receives data related to the movement inducing device 400 for inducing the movement of the pet and the selection of the exercise scenario from the user terminal 200.

In addition, the communication interface part 110 provides a control signal for operating the movement inducing device 400 to the movement inducing device 400 on the basis of the control of the user. Furthermore, by operating the movement inducing device 400, the communication interface part 110 receives data for a movement or a change in a biometric signal from the wearable device 300 worn on the pet according to the movement of the pet.

In addition, the communication interface part 110 receives coordinate information for a location changed according to the movement of the pet and receives coordination information for a current location of the movement inducing device 400 moved by the pet from the agent device 500.

In the virtual space virtually generating an actual living environment of the pet, the metaverse environment generation part 120 generates the metaverse environment by mapping temporal information and spatial information for the plurality of movement inducing device 400 existing in the virtual space and for the wearable device 300 worn on the pet through the avatars, and stores and manages the generated metaverse environment for each user in the database 600.

In addition, the metaverse environment generation part 120 receives location information of the movement inducing device 400 and the wearable device 300 worn on the pet from the agent device 500 through the communication interface part 110 when the induction of the movement of the pet is realized on the basis of the control of the user accessing the metaverse environment. Then, on the basis of the received location information, the avatars of the pet and the movement inducing device 400 at the corresponding location in the virtual space are mapped and displayed while being synchronized in real time.

By synchronizing and connecting the avatars of the virtual space with the actual living environment of the pet, a temporal change for a location of the pet and the movement inducing device 400 is capable of being updated in real time. That is, the living environment in which the pet lives and the virtual environment of the metaverse environment are temporally matched and synchronized.

At this time, the location information of the pet and the movement inducing device 400 are coordinate information that checks in which space the pet and the movement inducing device 400 are located among the spaces partitioned at the predetermined intervals, the coordinate information being checked on the basis of image information captured by the camera in the agent device 500 provided in the environment where the pet lives.

Accordingly, on the basis of the coordinate information of the pet and the movement inducing device 400 received from the agent device 500, the metaverse environment generation part 120 updates the avatars for the pet and the movement inducing device 400 in real time and displays the avatars at the corresponding location in the virtual space.

The pet monitoring part 130 monitors an action of the pet in the generated metaverse environment.

For example, movement information (a direction of a movement, a speed, and so on) of the action of the pet measured through the acceleration sensor and the gyro sensor in the wearable device 300 is received and coordinate information for the location of the pet checked from the agent device 500 is received, and the movement information and the coordinate information are displayed on the metaverse environment.

At this time, in the metaverse environment, the action of the pet is capable of being synchronized by updating the display of the location of the pet through the avatar rather than the image of the pet.

The pet exercise amount measuring part 140 measures the exercise amount of the pet by utilizing movement information provided from the wearable device 300, coordinate information for the location of the pet provided from the agent device 500, and so on.

At this time, the exercise amount of the pet is capable of being calculated by reflecting changes in coordinates and speed for the movement of the pet mapped through the avatar in the metaverse environment, values detected by the gyro sensor and the acceleration sensor that are provided in the wearable device 300, or a combination thereof.

Accordingly, in order to satisfy the desired target exercise amount on the basis of the exercise amount of the pet measured from the pet exercise amount measuring part 140, the user is capable of inducing the movement of the pet by operating the movement inducing device 400, thereby satisfying the exercise amount required for the pet.

The movement inducing part 150 operates the movement inducing device 400 on the basis of the control of the user accessing the generated metaverse environment. That is, when the user induces the movement of the pet in the metaverse environment, the exercise amount required for the pet is capable of being satisfied.

For example, the operation of the movement inducing part 150 is controlled so that vibration, sound, an image, smell, and so on are generated through the various movement inducing devices 400 such as the house of the pet, the toy, the play tool, the feed bucket, a microphone, the image display device, the speaker, the smell generator, and so on by a specific exercise scenario or the direct selection and control of the user, thereby inducing the attraction of the pet and exercising the pet.

Meanwhile, when a plurality of pets is existing in the same space, the movement inducing part 150 controls the operation of the movement inducing device 400 according to different scenarios for of the plurality of pets on the basis of the operation of the user accessing the metaverse environment, thereby being capable of inducing the movement for each pet.

For example, when a specific exercise scenario is selected for the plurality of pets, the movement inducing part 150 initially controls the operation of the movement inducing device 400 commonly applied in an exercise scenario that will be performed for each pet, so that the movement is capable of being induced while each pet reacts simultaneously.

After the operation of the movement inducing device 400 commonly applied is finished, the movement inducing part 150 separately controls the operation of the movement inducing device 400 according to an exercise scenario that is separately processed for each pet, so that each pet is capable of being separately moved according to the movement inducing of each movement inducing device 400. At this time, in order to smoothly induce each pet, the movement inducing part 150 is capable of being configured to operate the favorite toy or the play tool of the pet, or to operate the image of the user calling the name of the pet.

Here, an operation sequence of each movement inducing device 400 for inducing the movement of the plurality of pets is an example, and is capable of being changed.

The exercise scenario setting part 160 sets at least one exercise scenario for the pet movement induction, the exercise scenario including an operation sequence, an operation method, a reward providing of each movement inducing device 400 positioned in the virtual space. Furthermore, the exercise scenario setting part 160 stores and manages the set exercise scenario in the database 600.

At this time, in the exercise scenario, at least one objective such as weight loss, specific part training, simple exercise, and so on of the pet is capable of being set with reference to shapes and types of the movement inducing devices 400 disposed in the virtual space for each user in the metaverse platform 100.

In the customized exercise scenario setting part 161, after the user accessing the metaverse environment checks each movement inducing device 500 positioned in the virtual space, a customized exercise scenario in which the user directly selects the operation sequence, the operation method, the reward providing, and so on of each movement inducing device 400 is stored in the database 600 and is managed.

That is, the metaverse platform 100 automatically sets an exercise scenario according to the shapes and the types of each movement inducing device 400 positioned in the virtual space that is generated for each user, the objective of the exercise of the pet, and so on. Furthermore, the user is capable of determining an exercise scenario by directly customizing an exercise scenario.

The exercise scenario providing part 170 provides a specific exercise scenario that the user selects among a plurality of exercise scenarios set through the exercise scenario setting part 160 to the movement inducing part 150, or provides data for a customized exercise scenario that the user directly selects through the customized exercise scenario setting part 161 to the movement inducing part 150.

The exercise scenario assessing part 180 continuously tracks movement information of the pet received from the wearable device 300 and continuously tracks the movement of the pet in which the movement is induced by the specific exercise scenario or the customized exercise scenario on the basis of coordinate information for a location received from the agent device 500. Then, by summarizing the tracked results, the exercise scenario assessing part 180 assesses how much the exercise of the pet has been performed and which body part of the pet has been induced to exercise.

With reference to the exercise results of the pet assessed from the exercise scenario assessing part 180, the exercise scenario modifying part 190 modifies the exercise scenario or the customized exercise scenario for complementing the insufficient movement or the insufficient action of the pet, and provides the modified results to the user.

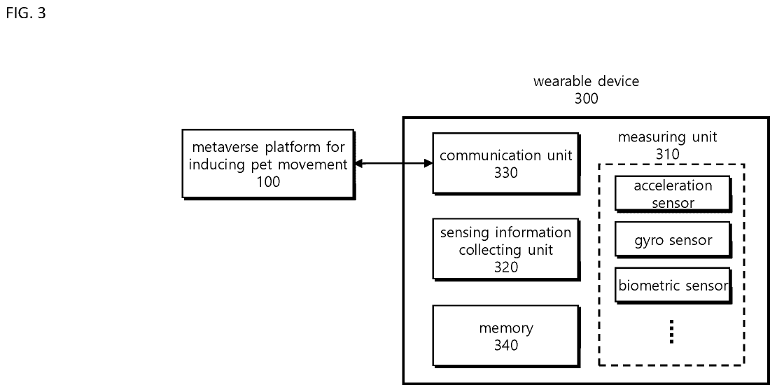

FIG. 3 is a view specifically illustrating a wearable device according to an embodiment of the present disclosure.

As illustrated in FIG. 3, the wearable device 300 includes a measuring unit 310, a sensing information collecting unit 320, a communicating unit 330, a memory 340, and so on.

The measuring unit 310 is capable of including a biometric sensor measuring biometric information such as a temperature, a pulse and so on by being in contact with a body part of the pet, an acceleration sensor measuring a movement and a direction of the corresponding pet, a gyro sensor, and so on.

The sensing information collecting unit 320 periodically collects sensing information of the pet measured from each sensor provided in the measuring unit 210, the sensing information including biometric information, movement information, or a combination thereof. Otherwise, the sensing information collecting unit 320 collects the sensing information in real time.

The communicating unit 330 performs a communication connection with the metaverse platform 100, and transmits the sensing information collected from the sensing information collecting unit 320 to the metaverse platform 100.

The memory 340 temporarily stores the sensing information detected through the measuring unit 310.

Meanwhile, although not illustrated in FIG. 3, when the wearable device 300 is used in the outdoor, a GPS receiver for checking the location of the pet is further capable of being included, and a vibration generator, a speaker, and so on are capable of being included so as to generate vibration or sound so that the pet is capable of looking around when the pet does not respond. Here, the GPS receiver is configured to check the location of the pet in an outdoor activity of the pet, rather than an indoor activity of the pet.

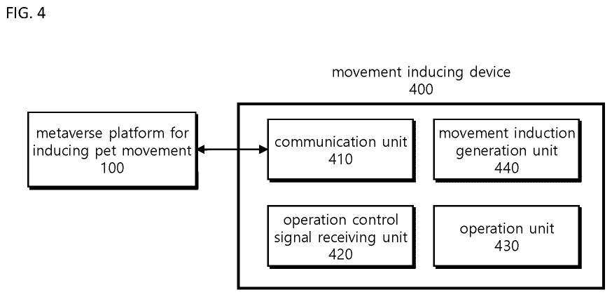

FIG. 4 is a view specifically illustrating a movement inducing device according to an embodiment of the present disclosure.

As illustrated in FIG. 4, the movement inducing device 400 includes a communication unit 410, an operation control signal receiving unit 420, an operation unit 430, a movement induction generation unit 440, and so on.

The communication unit 410 performs a communication connection with the metaverse platform 100, and receives compressed data from the metaverse platform 100, the compressed data including an operation control signal for operating the corresponding movement inducing device 400.

The operation control signal receiving unit 420 extracts the operation control signal by demodulating the compressed data received from the metaverse platform 100 through the communication unit 410, and transmits the operation control signal to the operation unit 430.

The operation unit 430 performs an operation of the movement induction generation unit 440 according to the operation control signal.

The movement induction generation unit 440 generates vibration, sound, an image, smell, or a combination thereof according to the control of the operation unit 430. For example, in the image display device, an image or a content of the user is played and displayed. Furthermore, in the toy, vibration or sound is generated.

Meanwhile, although not illustrated in FIG. 4, the movement inducing device 400 is further capable of including a GPS receiver for checking a location, and so on when the movement inducing device 400 is used in the outdoor as same as the wearable device 300.

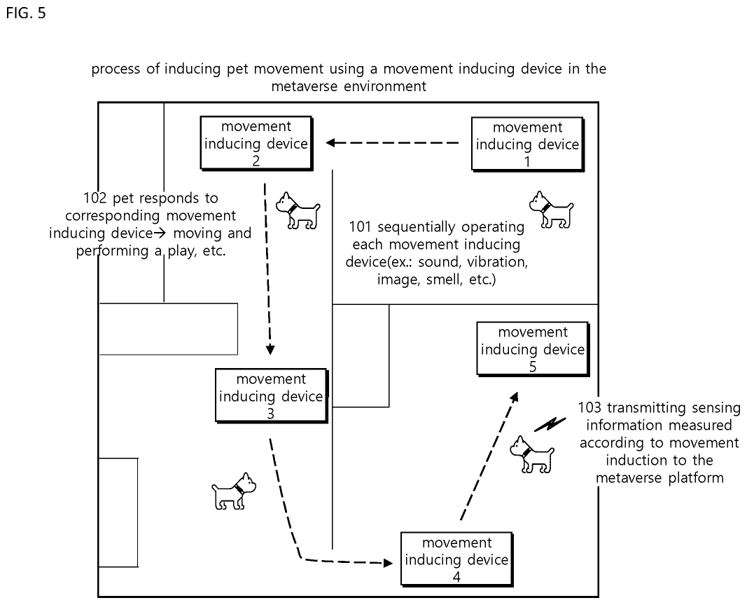

FIG. 5 is a view specifically illustrating a pet movement inducing process in the metaverse environment according to an embodiment of the present disclosure.

As illustrated in FIG. 5, when the user accesses the metaverse environment through the user terminal 200 in which the metaverse environment realizing the actual living space of the pet as the virtual space, the metaverse platform 100 allows the user to check information for an action or a movement of the pet positioned in the family house through the virtual space constructed for each user.

Next, the user directly selects the operation of the movement inducing device 400 in various shapes and types in which the movement inducing device 400 includes the toy, the play tool, the feed bucket, and so on that are provided in the family house, or the user selects a set exercise scenario according to a specific objective (for example, weight loss, specific part training, simple exercise, and so on). For example, as illustrated in FIG. 5, it is assumed that a movement inducing device 1 to a movement inducing device 5 are sequentially selected to perform a movement inducing.

In the metaverse platform 100, an operation control signal is generated such that the movement inducing devices 1 to 5 are operated at a predetermined time interval according to the direct selection or the exercise scenario. Then, the generated operation control signal is sequentially transmitted to the movement inducing devices 1 to 5 according to a predetermined sequence or a predetermined time.

Therefore, the movement inducing devices 1 to 5 are sequentially operated according to the operation control signal, and vibration, sound, an image, smell, and so on are generated 101.

Accordingly, as the movement inducing devices 1 to 5 are sequentially operated, the attraction of the pet is induced. Through this, the pet responds according to the operations of the movement inducing devices 1 to 5, and the pet performs the exercise while performing play and so on 102.

When the pet is moved according to the sequential operation of the movement inducing devices 1 to 5, information for the movement of the pet measured through the acceleration sensor, the gyro sensor, and so on, and biometric information such as a temperature, a pulse, and so on detected through the biometric sensor are output to the metaverse platform 100 from the wearable device 300. At the same time, the agent device 500 transmits coordinate information for the location of the pet moved by the induction of the movement inducing devices 1 to 5 checked by analyzing image information captured by the camera, coordinate information for the location of the movement inducing device capable of being moved among the movement inducing devices 1 to 5, or the like to the metaverse platform 100 at 103.

Accordingly, as the metaverse platform 100 updates and displays the avatars for the pet and the movement inducing devices 1 to 5 in real time on the basis of the coordinate information received from the agent device 500, the user is capable of checking the current locations of the pet and the movement inducing device through the virtual space, and is capable of checking whether the movement of the pet is effectively performed.

In addition, the metaverse platform 100 continuously tracks the movement of the pet on the basis of the coordinate information received from the agent device 500, so that the metaverse platform 100 is capable of assessing whether the movement of the pet is correctly performed according to the exercise scenario.

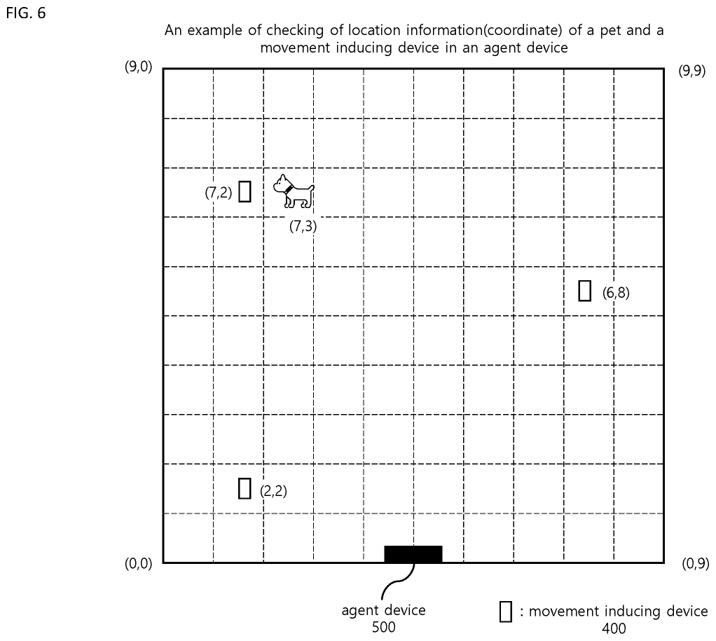

FIG. 6 is a view illustrating checking of location information (coordinate) of a pet and the movement inducing device in the metaverse environment according to an embodiment of the present disclosure.

As illustrated in FIG. 6, the metaverse platform 100 is capable of vertically and horizontally partitioning the actual living space of the pet realized in the virtual space at predetermined intervals, and is capable of assigning coordinates to each space. For example, as illustrated in FIG. 6, 100 spaces are partitioned, and coordinates from (0,0) to (9,9) is capable of being set for each space.

In a state in which the actual living space of the pet is partitioned into the plurality of spaces and coordinates are set, the agent device 500 is capable of checking the coordinates for the location or the pet or the movement inducing device 400 with reference to image information captured by the camera.

That is, the agent device 500 analyzes image information for the living space of the pet captured at a current time and image information captured at a previous unit time, and analyzes to which space the pet and the movement inducing device 400 have been moved. Then, coordinates set at a specific space where the pet and the movement inducing device 400 are moved on the basis of the analyzed results are capable of being determined as the space where the pet and the movement inducing device 400 are located.

For example, as illustrated in FIG. 6, when the spaces where the pet and each movement inducing device 400 are located are checked resulting from analyzing the image information captured by the camera, the agent device 500 checks that the pet is located at coordinates (7,3) and each movement inducing device 400 is located at coordinates (7,2), (6,8), and (2,2), and the agent device 500 transmits coordination information for the pet and each movement inducing device 400 to the metaverse platform 100.

In addition, in the metaverse platform 100, on the basis of the coordinate information of the pet and the movement inducing device 400, the avatars for the pet and the movement inducing device 400 are capable of being updated in real time and are capable of being displayed at the corresponding location in the virtual space.

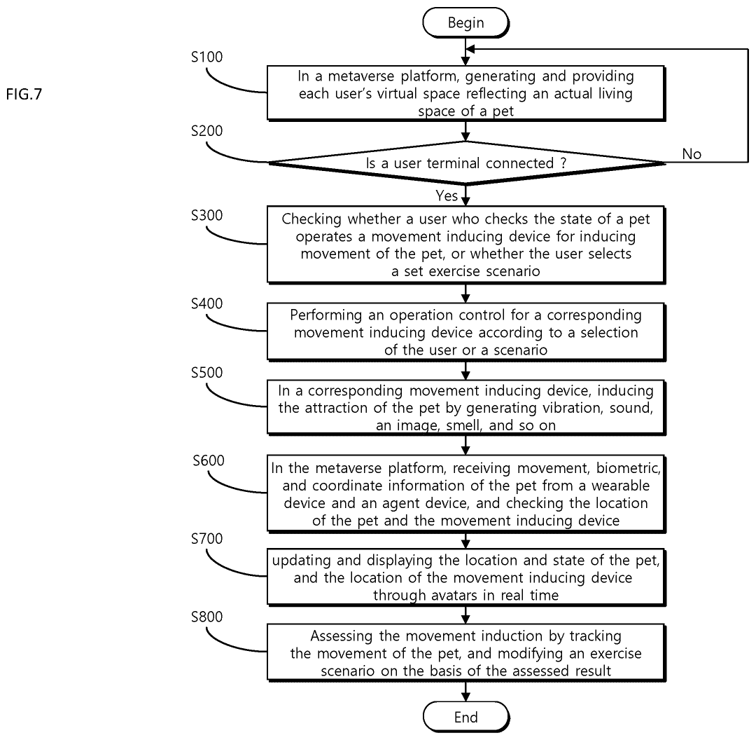

Next, an embodiment of a pet movement inducing method in the metaverse environment according to the present disclosure constituted as described above will be described in detail with reference to FIG. 7. At this time, a sequence of each process according to the method of the present disclosure is capable of being changed by a use environment or those skilled in the art.

FIG. 7 is a flowchart specifically illustrating an operation process of a pet movement inducing method in the metaverse environment according to an embodiment of the present disclosure.

As illustrated in FIG. 7, the metaverse platform 100 generates and provides a virtual space for each user, the virtual space reflecting an actual living space of a pet S100. That is, a metaverse environment in which information for the wearable device 300 worn on the pet and information for the movement inducing device 400 inducing an action of the pet existing in the virtual space are mapped through avatars is provided.

In addition, the metaverse platform 100 determines whether the user accesses the metaverse platform 100 by executing an application program installed in the user terminal 200 of the user S200. Then, when the access of the user terminal 200 is checked, information for an action or a movement of the pet located in the family house through the virtual space is provided to the user terminal 200 so that user is capable of checking the information.

In addition, the metaverse platform 100 checks whether the user directly selects the operation of the movement inducing device 400 in various shapes and types in which the movement inducing device 400 includes the toy, the play tool, the feed bucket, and so on, or whether the user selects a set exercise scenario according to an objective such as weight loss of the pet, a simple exercise, and so on S300.

Next, the metaverse platform 100 generates an operation control signal for operating the movement inducing device 400 according to the direct selection of the user or the exercise scenario, and transmits the generated operation control signal to the corresponding movement inducing device 400 according to a predetermined sequence or a predetermined time S400. At this time, the operation control signal is capable of including identification information of each movement inducing device 400, and a specific movement inducing device 400 is only operated on the basis of the identification information.

The movement inducing device 400 induces the attraction of the pet by generating vibration, sound, an image, smell, and so on according to the operation control signal received from the metaverse platform 100 through the S400 process S500.

Accordingly, when the pet is moved by the movement inducing device 400 and the pet performs the movement, the metaverse platform 100 receives coordinate information for locations of the pet and the movement inducing device 400 through a network, the coordinate information being obtained by analyzing information for the movement of the pet measured from the wearable device 300 and image information captured by the camera in the agent device 500. Then, the metaverse platform 100 checks the location of the pet and the movement inducing device 400 S600.

Next, with reference to the movement information (that is, a direction, a speed, and so on) received from the wearable device 300 and the coordinate information received from the agent device 500, the metaverse platform 100 updates the avatars for the pet and the movement inducing device 400 in real time and displays the avatars at the corresponding location in the virtual space S700.

In addition, the metaverse platform 100 continuously tracks the movement of the pet displayed according to the S700 process, assesses whether the movement of the pet is normally performed according to the exercise scenario or the movement inducing of the movement inducing device 400 directly selected by the user on the basis of the tracked results, and modifies or complements the exercise scenario on the basis of the assessed results S800.

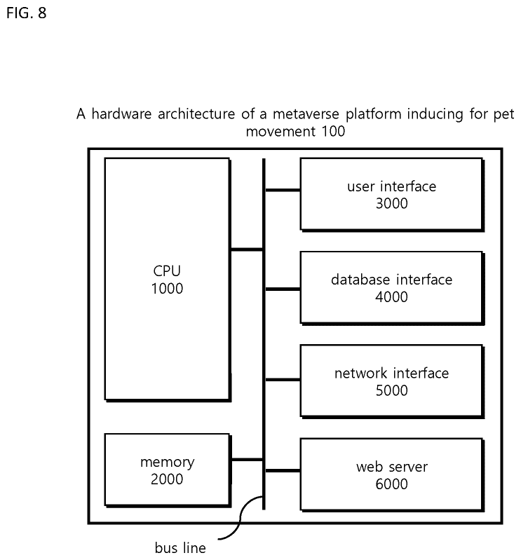

FIG. 8 is a view illustrating an example of a hardware architecture of a computer (server) realizing the metaverse platform for inducing the pet movement, according to an embodiment of the present disclosure.

As illustrated in FIG. 8, a hardware structure of the metaverse platform 100 for inducing the movement of the pet includes a central processing unit 1000, a memory 2000, a user interface 3000, a database interface 4000, a network interface 5000, a web server 6000, and so on.

The user interface 3000 uses a graphical user interface (GUI), and provides an input interface and an output interface to the user.

The database interface 4000 provides an interface between the database and the hardware structure. The network interface 5000 provides a network connection between devices that the user owns.

The web server 6000 provides a means for the user to access the hardware structure through the network. Most of the users are capable of using the metaverse platform 100 by accessing the web server 6000 remotely.

The configuration as described above and each process of the method are capable of being realized as computer readable codes on a computer readable recording medium, or are capable of being transmitted through a transmission medium. The computer readable recording medium is a data storage device that can store data which can be read by a computer system.

An example of the computer readable recording medium includes a read-only memory (ROM), a random-access memory (RAM), a compact disc (CD)-ROM, a digital versatile disc (DVD), a magnetic tape, a floppy disk, and an optical data storage device, but is not limited thereto. The transmission medium is capable of including carrier waves transmitted through the Internet or various types of communication channels. In addition, the computer readable recording medium is also capable of being distributed over network coupled computer systems so that the computer readable code is stored and executed in a distributed manner.

In addition, at least one component applied in the present disclosure is capable of including a processor such as a central processing unit (CPU), a microprocessor, and so on performing each function, or is capable of being realized of the processor. Furthermore, two or more of the components are capable of being coupled as a single component and are capable of performing all operations and functions for the two or more of the components. In addition, a function of a part of at least one component is capable of being performed by another component among the components. In addition, a communication between the components is capable of being performed by a bus (not illustrated).

Accordingly, in the present disclosure, since the user is capable of accessing the metaverse environment and is capable of performing an interaction with a corresponding pet by utilizing tools in various shapes and types including the toy or by the automatically set scenario, the exercise amount required for the pet is satisfied, so that the body of the pet is capable of being kept healthy.

In addition, in the present disclosure, voice, image, smell, and so on of the user are utilized so as to request a specific action to the pet, and a reward for the specific action is provided to the pet, so that exercise efficiency of the pet is capable of being increased.

In addition, in the present disclosure, the movement of the pet according to the interaction with the user is continuously tracked, and a program capable of complementing an insufficient movement or an insufficient action of the pet is capable of being proposed through the tracked results, so that an advanced movement is capable of being induced for each pet.

As described above, the present disclosure has been described with reference to the embodiment illustrated in the drawings, but this is only exemplary, and those skilled in the art to which the art pertains will understand that it is possible to have various modifications and other equivalent embodiments. Therefore, the technical protection scope of the present disclosure should be determined by the following claims.

What is claimed is:

1. A system for inducing pet movement in metaverse environment, the system comprising:
a metaverse environment generation part configured to generate a metaverse environment by mapping temporal and spatial information for a wearable device worn on a pet and for a movement inducing device inducing an action of the pet existing in a virtual space for a living space of the pet; and
a movement inducing part configured to operate the movement inducing device on the basis of a control of a user accessing the generated metaverse environment,
wherein the exercise amount required for the pet is satisfied by the user who induces the pet movement in the metaverse environment.

2. The system of claim 1, wherein the metaverse environment generation part is further configured to:
receive location information for the wearable device of the pet and the movement inducing device,
map the received location information to avatars of the pet and the movement inducing device at corresponding locations in the virtual space, and
update a temporal change of the location information in real time, thereby synchronizing and connecting the avatars in the virtual space to a living environment where the pet actually lives.

3. The system of claim 2, wherein the location information of the pet and the movement inducing device is coordinate information that checks in which space the pet and the movement inducing device are located among spaces partitioned at predetermined intervals, the coordinate information being checked on the basis of image information captured by a camera in an agent device provided in the living environment where the pet lives, and
wherein the metaverse environment generation part is configured to update the avatars for the pet and the movement inducing device in real time and to display the avatars at the corresponding location in the virtual space on the basis of the coordinate information of the pet and the movement inducing device received from the agent device.

4. The system of claim 1, wherein the system further comprises: a pet monitoring part configured to monitor the action of the pet in the metaverse environment,
wherein the action of the pet is synchronized by updating location information of the pet in the metaverse environment through the avatars rather than the image of the pet.

5. The system of claim 1, the system further comprises: a pet exercise amount measuring part configured to measure the exercise amount of the pet, and
wherein the movement of the pet is induced by the user in the metaverse environment so that a target exercise amount of the pet is reached on the basis of the measured exercise amount of the pet, thereby satisfying an exercise amount required for the pet, and
wherein the exercise amount of the pet is calculated by applying changes in coordinates and speeds for the movement of the pet in the metaverse environment, values detected from a gyro sensor and an acceleration sensor, or a combination thereof.

6. The system of claim 1, wherein the movement inducing device comprises a house, a toy, play tool, a feed bucket, a microphone, an image display device, a speaker, or a smell generator, and
wherein an operation of the movement inducing device is performed by vibration, sound, an image, smell, or a combination thereof.

7. The system of claim 1, wherein the system comprises: an exercise scenario setting part configured to set and store at least more than one scenario for the pet movement induction, including an operating sequence, an operating method, or a reward offering for each operation of at least more than one movement inducing device located in the virtual space provided for each user; and
an exercise scenario providing part configured to provide information for a specific scenario that the user selects among the set scenarios to the movement inducing part,
wherein the movement inducing part is further configured to control operating at least one of the movement inducing device according to the specific scenario that the user selects.

8. The system of claim 7, wherein the system further comprises: an exercise scenario assessing part configured to assess which body part is induced to exercise by continuously tracking the movement of the pet in which the movement is induced by the specific scenario according to the location information received from the wearable device; and
an exercise scenario modifying part configured to modify a scenario so as to complement an insufficient movement and an insufficient action of the pet on the basis of the assessed results.

9. The system of claim 8, wherein the system further comprises: a customized exercise scenario setting part configured to set and store a customized scenario comprising the operation sequence, the operation method, or the reward offering of at least one movement inducing device located in the virtual space according to a direct selection of the user accessing the metaverse environment, and
wherein the exercise scenario modifying part is configured to modify the customized scenario on the basis of the assessed results of the exercise scenario assessing part for the movement of the pet according to the customized scenario.

10. The system of claim 1, wherein the system is further configured to:
control an operation such that the movement inducing device is operated through the movement inducing part according to different scenarios for a plurality of pets on the basis of an operation of the user accessing the metaverse environment; and
initially control the operation of the movement inducing device commonly applied among scenarios performed for each of the plurality of pets, and then control a remaining operation of the movement inducing device separately performed.

11. A method for inducing pet movement in metaverse environment, the method comprising:
generating a metaverse environment by mapping temporal and spatial information for a wearable device worn on a pet and for a movement inducing device inducing an action of the pet existing in a virtual space for a living space of the pet; and
inducing a movement by operating the movement inducing device on the basis of a control of a user accessing the generated metaverse environment,
wherein the exercise amount required for the pet is satisfied by inducing the pet movement in the metaverse environment.

12. The method of claim 11, wherein the generating of the metaverse environment further comprises:
receiving location information for the wearable device of the pet and the movement inducing device,
mapping the received location information to avatars of the pet and the movement inducing device at corresponding locations in the virtual space, and
updating a temporal change of the location information in real time, thereby synchronizing and connecting the avatars in the virtual space to a living environment where the pet actually lives.

13. The method of claim 12, wherein the location information of the pet and the movement inducing device is coordinate information that checks in which space the pet and the movement inducing device are located among spaces partitioned at predetermined intervals, the coordinate information being checked on the basis of image information captured by a camera in an agent device provided in the living environment where the pet lives, and
wherein the generating of the metaverse environment is configured to update the avatars for the pet and the movement inducing device in real time and to display the avatars at the corresponding location in the virtual space on the basis of the coordinate information of the pet and the movement inducing device received from the agent device.

14. The method of claim 11, wherein the method further comprises:
monitoring the action of the pet in the metaverse environment,
wherein the action of the pet is synchronized by updating location information of the pet in the metaverse environment through the avatars rather than the image of the pet.

15. The method of claim 11, wherein the method further comprises:
measuring the exercise amount of the pet,
wherein the movement of the pet is induced by the user in the metaverse environment so that a target exercise amount of the pet is reached on the basis of the measured exercise amount of the pet, thereby satisfying an exercise amount required for the pet, and
wherein the exercise amount of the pet is calculated by applying changes in coordinates and speeds for the movement of the pet in the metaverse environment, values detected from a gyro sensor and an acceleration sensor, or a combination thereof.

16. The method of claim 11, the method further comprises:
setting an exercise scenario by setting and storing at least more than one scenario for the pet movement induction, including an operating sequence, an operating method, or a reward offering for each operation of at least more than one movement inducing device located in the virtual space provided for each user; and
providing the exercise scenario by providing information for a specific scenario that the user selects among the set scenarios to the inducing of the movement,
wherein the inducing of the movement comprises controlling to operate at least one of the movement inducing device according to the specific scenario that the user selects.

17. The method of claim 16, wherein the method further comprises:
in the system inducing the movement of the pet, assessing the exercise scenario by assessing which body part is induced to movement by continuously tracking the movement of the pet in which the movement is induced by the specific scenario according to the location information received from the wearable device; and
modifying the exercise scenario by modifying the scenario so as to complement an insufficient movement and an insufficient action of the pet on the basis of the assessed results.

18. The method of claim 17, wherein the method further comprises:
setting and storing a customized scenario comprising the operation sequence, the operation method, or the reward offering of at least one movement inducing device located in the virtual space according to a direct selection of the user accessing the metaverse environment,
wherein the modifying of the exercise scenario is configured to modify the customized scenario on the basis of the assessed results of the assessing of the exercise scenario for the movement of the pet according to the customized scenario.

19. The method of claim 11, wherein the method is further configured to:
control an operation such that the movement inducing device is operated through the inducing of the movement according to different scenarios for a plurality of pets on the basis of an operation of the user accessing the metaverse environment; and
initially control the operation of the movement inducing device commonly applied among scenarios performed for each of the plurality of pets, and then control a remaining operation of the movement inducing device separately performed.
